(19) United States
(12) Patent Application Publication
(10) Pub. No.: US 2021/0085968 A1
(43) Pub. Date: Mar. 25, 2021

(54) BIO-FIELD ARRAY AND METHODS OF USE

(71) Applicant: MCP IP LLC, Dover, DE (US)

(72) Inventor: Marcy C. Purnell, Hernando, MS (US)

(73) Assignee: MCP IP LLC, Dover, DE (US)

(21) Appl. No.: 16/603,638

(22) PCT Filed: Apr. 10, 2018
(86) PCT No.: PCT/US2018/026932

§ 371 Date: Oct. 8, 2019

Related U.S. Application Data
(60) Provisional application No. 62/483,889, filed on Apr. 10, 2017.

(51) Int. Cl. A61N 1/32; A61N 1/20

(52) U.S. Cl. CPC A61N 1/327; A61N 1/20

(57) ABSTRACT

Methods of using an apparatus for generating dielectrophoretic electromagnetic fields (dc-DEP EMF) to enhance cell polarity through the application of dc-DEP EMF force to stabilize Hyaluronan Mediated Motility Receptor (HMMR) and differentially modulate its expression in cancerous and noncancerous cells. A significant down regulation of HMMR when coupled with the decreased cell growth/mitosis and decreased mitotic spindle formation in treated human breast carcinoma cells indicating a decreased cell motility/migration/metastasis occurs when the cancerous cells are exposed to the dc-DEP EMF force. An improved apparatus for generating dielectrophoretic electromagnetic fields comprising crystals in the center of the orb for enhanced bio-field, a power supply toroidal flow of direct current to the orb, sensing capabilities to measure impedance, and customization of electromagnetic signals emitted by the apparatus and the power supply.

### CROSS REFERENCE TO RELATED APPLICATIONS

This application claims priority to U.S. Provisional Application No. 62/483,889 filed on Apr. 10, 2017, the contents of which are incorporated herein by reference in its entirety.

### FIELD OF THE INVENTION

The present invention is generally directed toward improvements of an apparatus for generating dielectrophoretic electromagnetic fields and methods of using an apparatus for generating dielectrophoretic electromagnetic fields.

### BACKGROUND OF THE INVENTION

In 2016 there were an estimated 246,660 new cases of invasive breast cancer and 61,000 new cases of non-invasive breast cancer diagnosed in women in the United States (ASCO, 2016). About 5-10% of breast cancers can be linked to genetic mutations that are inherited (Greenberg, 2006). The most common inherited genetic mutations occur in the BRCA1/2 genes (Greenberg, 2006). Women with the BRCA1 mutation have a 55-65% lifetime risk of developing breast cancer while women with the BRCA2 mutation have a 45% increased risk (Chen & Parmigiani, 2007). Breast cancer tends to develop more often in younger women who test positive for these inherited mutations. An increased risk of ovarian and other cancers in both men and women is also associated with these inherited mutations (Lynch, Casey, Snyder, Bewtra, Lynch, Butts & Godwin, 2009).

Women who test positive for BRCA1 mutations and are treated for Stage I or II breast cancer with bilateral mastectomy are less likely to die from breast cancer than women who are treated with unilateral mastectomy (Metcalf, et al., 2014). Also, the rates of contralateral prophylactic mastectomy (CPM) in women with no inherited mutations and unilateral breast cancer are increasing despite the fact that the majority of women achieve no oncologic benefit from the CPM (Fisher, 2016). There are many reasons women chose CPM, the predominant theme for many is often peace of mind.

Hyaluronan mediated motility receptor (HMMR), also known as RHAMM (Receptor for Hyaluronan Mediated Motility), is a non-integral cell surface hyaluronan receptor (CD168) and intracellular protein that is encoded by the HMMR gene and promotes cell motility in culture (Shigeishi, Higashikawa & Takechi, 2014; Hamilton et al., 2007). This protein is located in the cytoskeleton and centrosome and has been linked to modulation of growth factor receptors, regulation of cell signaling pathways and mitotic spindle assembly (Maxwell, McCarthy & Turley, 2008). RHAMM has initiated multiple motogenic signaling pathways such as Ras, pp60-c-SRC ERK1 and ERK2 (Wang et al., 1998). It has also performed motogenic and invasive functions that are similar to and may actually enhance CD44 (Hamilton et al., 2007). While RHAMM has been less studied than CD44 with regards to cancer metastasis, it is just as important. Once a metastatic lesion is formed, increased RHAMM will also partner with CD44 to promote angiogenesis (Savani et al., 2001; Hamilton et al., 2007). Extracellular HMMR forms a complex with CD44 which then combines with hyaluronan and activates intracellular signaling pathways such as extracellular signal-regulated kinase (ERK), that regulate tumor cell proliferation and migration (Tilghman, Wu & Sang, 2014). Also, RHAMM is a mitotic-spindle/hyaluronan binding cytoplasmic and nuclear protein (Nickel, 2005) that shows interaction with interphase microtubules, centrosomes and the mitotic spindle formation revealing its multiple functions across multiple cell compartments (Assmann, Jenkinson, Marshall, Hart, 1999).

RHAMM, a protein encoded by HMMR, is not highly expressed in normal tissues but has been known to play a significant role in tumorigenesis, genomic instability and cancer progression. Spindle forming functions of RHAMM are inhibited by the breast/ovarian tumor suppressor gene BRCA1. RHAMM is expressed in breast tissue and together forms a complex with BRCA1 and other proteins (BRCA1-AURKA-RHAMM-TPX2-TUBG1) that are known to regulate mammary cell polarization that is necessary for mammary stem cell fate. BRCA1 has also participated in the maintenance of apicobasal membrane polarity. Cell polarity differs in cancerous, injured and proliferating cells where transmembrane potential is ˜<−30 mV, while noncancerous cells have a resting potential of ˜>−70 mV (Zhou and Uesaka, 2006; Yang and Brackenbury, 2013; Levin, 2014; Lobikin et al., 2012). The loss of tissue polarity, due to BRCA1 mutation or other possible factors that change the cell microenvironment, is an early hallmark of breast cancer (Maxwell, 2011; Blanco et al., 2015). When a BRCA1 mutation occurs, the loss of cell polarity and lack of silencing of HMMR from the BRCA1 gene is a contributing factor to tumor development, progression and metastasis, as shown in FIG. 3B. Also, in the absence of a BRCA1 mutation, the overexpression of RHAMM in cancers correlates with increased metastasis and poor outcomes showing that this protein may be highly influential in both inherited and non-inherited forms of breast cancer (Auvinen, Tammi, Parkkinen, Tammi, Agren, et al., 2000; Blanco et al., 2015).

Terminal differentiation of mammary epithelial cells is thought to be mediated by both BRCA1 activation and RHAMM degradation (Maxwell, 2011; Blanco et al., 2015). Interestingly, an increased cell motility/metastasis is seen through a number of different pathways with an increased RHAMM (Maxwell, 2011; Blanco et al., 2015). RHAMM is unconventionally exported to the cell surface in response to stimuli such as wounding and cytokine release. While this may be an innate compensatory response that is designed to facilitate wound healing and the necessary cell migration that is needed for healing and repair, it can also have devastating consequences when this response leads to cell migration/metastasis of a cancerous cell (Tolg, McCarthy, Ariang, Yazdani & Turley, 2014; Purnell et al., 2016). Triple negative and HER2 subtype breast cancers have been found to have increased expression of RHAMM and patients with this increased expression have often suffered increased metastasis and ultimately poor outcomes and decreased survival (Edward, Gillan, Miche & Tammi, 2005). Thus, depolarization of the human mammary tissue as is often seen in wounds and inflammatory microenvironments also occurs in the absence of the BRCA1 mutation and possibly activate HMMR expression and other cell migratory pathways (Furuta, 2005; Purnell et al., 2016). The mechanisms that link cell migration/wound healing and cell migration/cancer metastasis pose an example of how challenging the development of side effect free cancer treatments can be (Schafer & Werner, 2008).

A direct current (de) dielectrophoretic (DEP) electromagnetic field (EMF) force that is generated by the water treatment apparatus taught in U.S. Pat. No. 6,555,071 B2 (also referred to herein as the “bio-field array” or “BFA” apparatus), incorporated herein by reference, enhances cell polarity through the effect of bio-chloride (_{b}Ct) on diamagnetic anisotropy taught in PCT/US2018/014238, incorporated herein by reference (Purnell et al., 2016). This bio-field array apparatus (3FA) has been sold commercially for use in baths/footbaths since 1996 and consumers claim it provides health benefits ranging from lowering blood pressure to faster wound healing. These experiments investigate how exposing a dilute saline solution to the dc-DEP EMF force generated by the BFA and then using this saline to reconstitute growth media of MDA-MB-231 and MCF-10A cells affects cell growth over time and ultimately gene expression in vitro. These findings of dc-DEP EMF force driven enhanced cell polarity were found to show a significant growth inhibition in the human breast carcinoma without showing a growth inhibition in the human breast epithelial cells (Purnell et al., 2016). Apicobasal polarity is a unique finding in epithelial cells since it refers to the polarity interplay between the apical membrane and the basolateral membrane, separated by tight junctions (Khursheed & Bashyam, 2014). This polarity is an important part of cytoskeletal reorganization, WNT signaling, TGFB, integrin-mediated signaling and differentiation (Royer & Lu, 2011). Since BRCA1 mutations are thought to lead to loss of cell polarity and ultimately increased HMMR (increased migration/metastasis) expression (Furuta et al., 2005), the ability to enhance cell polarity with this dc-DEP EMF force generated by the BFA apparatus offers another way to stabilize cell polarity in both the absence of an intact BRCA1 influence as well as other adverse conditions of the cell microenvironment. This polarity stabilization not only decreases the risk of the developing cancer and subsequent metastasis with these inherited genetic mutations, but also helps to reduce these findings in other genetically driven aggressive cancers (Lobikin, Chernet, Lobo & Levin, 2012).

The development of electromagnetic field therapies and their use over the last century in various areas of medicine, has been slowed and often halted primarily by the lack of scientifically-derived, evidence-based knowledge of the mechanism of action and/or particle trajectory identification at the cellular level. (B. Strauch, C. Herman, R. Dabb, L. J. Ignarro & A. A. Pilla, Aesthet Surg J. 29, 2009). Thus, a need exists to determine therapeutic uses for the BFA apparatus and ways to improve the apparatus functionality.

### SUMMARY OF THE INVENTION

The present invention is directed to methods of using an apparatus for generating dielectrophoretic electromagnetic fields (dc-DEP EMF) to enhance cell polarity through the application of dc-DEP EMF force to stabilize Hyaluronan Mediated Motility Receptor (HMMR) and differentially modulate its expression in cancerous and noncancerous cells. A significant down regulation of HMMR when coupled with the decreased cell growth/mitosis and decreased mitotic spindle formation in treated human breast carcinoma cells indicates a decreased cell motility/migration/metastasis occurs when the cancerous cells are exposed to the dc-DEP EMF forte.

Also provided herein is an improved apparatus for generating dielectrophoretic electromagnetic fields in solution comprising at least one of the following improvements: crystals or other precious stones in the center of the orb for enhanced bio-field, a power supply toroidal flow of direct current to the orb, sensing and feedback capabilities to measure impedance, and customization of electromagnetic signals emitted by the apparatus and the power supply.

### BRIEF DESCRIPTION OF THE DRAWINGS

Further advantages of the invention will become apparent by reference to the detailed description of preferred embodiments when considered in conjunction with the drawings:

FIGS. 1A-1B depict bar graphs showing cell growth over time of MDA-MB-231 and MCF-10A cells in control and treated media. Control and treated media were reconstituted daily with dc-DEP EMF control/treated water and replaced daily. Wells were counted in triplicate daily with three repeats. Due to lack of normality, growth rates were analyzed with the Mann-Whitney U-test (p<0.05) and error bars are +/−s.e.m. Individually, FIG. 1A shows human 26 breast carcinoma with Z-Score of 4.3978; p=0.0001; U-value is 22.5; CV-99. n=36, 42, 36, 42. FIG. 1B shows human breast epithelial cells with Z-score 0.8701; p=0.3843; U-value is 134; n=36, 42,42, 42. Data are normal and z-value can be used.

FIGS. 2A-2B depict mitosis/mitotic spindle formation of MDA-MB231 cells in control (FIG. 2A) and treated (FIG. 2B) media after 24 hours of growth in control and treated media at 100× magnification. Cells were fixed and stained with DAPI to visualize the nuclei and then with anti-tubulin antibody followed by a rhodamine-conjugated secondary antibody for fluorescent microscopy analyses. Note the mitotic spindle formation that is occurring in the control human breast carcinoma (FIG. 2A) and note the lack of mitotic spindle formation with down-regulation of HMMR in the treated human breast carcinoma (FIG. 2B). Upon analysis, there were approximately 20 percent of the control cells undergoing some stage of mitosis (mitotic index) while there were 0 percent of the treated cells undergoing mitosis (n=36, n=12, n=36).

FIGS. 3A-3C depict interaction of BRCA1/HMMR in cell polarization. Individually, FIG. 3A depicts polarized cell-BRCA1-AURKA-RHAMM-TPX2-TUBG1 complex holds cell polarity and RHAMM stable. FIG. 3B depicts depolarized cell-BRCA1 mutation present which leads to up-regulation of HMMR and activation of migration transduction pathways. FIG. 3C depicts diamagnetically stabilized cell dc-DEP EMF diamagnetically stabilizes cell polarity and holds HMMR in check in cancer cell.

FIG. 4 depicts an embodiment of the bio-field array apparatus.

FIGS. 5A-5B depict oscilloscope readings off of the bio-field array apparatus. Individually, FIG. 5A depicts enhanced/modulated dB/Hz signaling (left), and FIG. 5B depicts dB/Hz without modulation of the improved design. In FIG. 5A, the first signal on the enhanced array appears to be 32 dB 125 Hz versus 29 dB 125 Hz on the standard array. The second signal on the enhanced array appears to be 20 dB at 250 Hz versus 19 dB at 250 Hz. The fourth signal appears to be 12 dB at 500 Hz versus no detectable reading on the standard array at 500 Hz.

FIGS. 6A-6B depict sound (dB/Hz) traveling through red blood cells creating stress that leads to transcriptional and post transcriptional level responses. Individually, FIG. 6A depicts enhanced array signal of round (smooth) biconcave disc shapes of the red blood cells 48 hours post-BFA treatment. FIG. 6B shows standard array signal (without modifications) with irregularities in the membrane, as indicated by the arrows.

FIG. 7 depicts an embodiment of the bio-field array apparatus.

### DETAILED DESCRIPTION

The following detailed description is presented to enable any person skilled in the art to make and use the invention. For purposes of explanation, specific details are set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

### Potential Adjunct Cancer and Cancer Metastasis Treatments

As previously discussed, mechanisms that regulate cancer cell metastasis are often intricately linked to mechanisms that control cell migration in wound repair. Hyaluronan Mediated Motility Receptor (HMMR) encodes a receptor for hyaluronan-mediated motility (RHAMM), a non-integral cell surface hyaluronan receptor and intracellular protein that promotes mitotic spindle formation and cell motility. R HAMM increased expression in both cancers and wounds and when cancers show increased RHAMM expression poor outcomes have occurred. Therefore, RHAMM contributes to both natural healing mechanisms and cancer cell pathology. RHAMM is expressed in breast tissue and forms a polarity normalizing complex with BRCA1. Mutations of BRCA1 have been associated with a loss of apicobasal cell polarity along with a subsequent increased expression of R HAMM. Thus, the data described herein teaches a new adjunct treatment as well as an alternative to CPM and other surgical interventions for patients with inherited and non-inherited aggressive carcinomas.

In the study, a human breast carcinoma cell line maintained in media prepared with a dilute saline solution that had been exposed to a dielectrophoretic (DEP) electromagnetic field (EMF) generated by 3 amperes of direct current (de) to an apparatus housing an array of conductive rings. This invention shows a significant inhibition of growth of human breast carcinoma, MDA-MB-231 cells when they were maintained in media prepared with dc-DEP EMF force treated dilute saline while no growth inhibition occurred when the same cells were maintained in identically prepared, but untreated media. Importantly, no growth inhibition was observed in human epithelial, MCF-10A cells when grown in either treated or untreated media. Also, mitotic spindle formation was inhibited in the human breast carcinoma when they were grown in dc-DEP EMF force treated media. To determine if any gene expression changes contributed to the selective growth inhibition and absent mitotic spindle formation in the human breast carcinoma grown in the treated media, microarray analysis was employed and found that there was large-scale transcriptional reprogramming of the tumor cells grown in the treated media with over 1,000 genes up- or down-regulated over 2-fold, whereas the non-cancerous MCF-10A cells showed relatively modest changes in gene expression. Of the genes affected in the MDA-MB-231 cells, there was a significant down regulation of HMMR. The ability to enhance cell polarity through the application of this dc-DEP EMF force offers another way to stabilize HMMR and differentially modulate its expression in cancerous and noncancerous cells.

### Experimental Methods and Design

### Cells and Cell Culture Medias

Human MDA-MB-231 triple-negative breast carcinoma and human MCF-10A breast epithelial cells were obtained from the American Type Culture Collection. The MDA-MB-231 cells were maintained in high glucose Dulbecco's Modified Eagles Medium (DMEM, Lonza #12-604Q) containing 10% fetal bovine serum (FBS; Atlanta Biologicals). MCF-10A cells were maintained in DMEM/F12 (Invitrogen cat. #11330-032) supplemented with 20 ng/ml epidermal growth factor (PeproTech cat. #100-47), 0.5 mg/ml hydrocortisone (Sigma-Aldrich cat. #H-0888), 100 ng/ml cholera toxin (Sigma-Aldrich cat. #C-8052), 10 μg/ml insulin (Sigma-Aldrich cat. #I-1882) and 5% horse serum (Invitrogen cat. #11330-032).

To prepare treated and control DMEM, 10×DMEM (Sigma cat. #D2429) was diluted 9:1 with a hypotonic saline solution that had been treated for 30 minutes at 3 amperes of de with the dc-DEP EMF apparatus, or with an aliquot of the same solution prior to treatment with the apparatus. The hypotonic saline solution consisted of 3 mM NaCl prepared using laboratory-grade deionized water and molecular biology-grade NaCl (Promega cat. #V4221). Complete treated and control DMEM was supplemented with 0.004 gm/L folic acid (Sigma-Aldrich cat. #F8758-5G), 4000 mg/L glucose (Sigma-Aldrich cat. G7021-100G), 0.584 gm/L glutamine (Sigma-Aldrich cat. #G7513) and 3.7 gm/L sodium bicarbonate (Biowhittaker cat. #15-6131) and filtered through a 0.45 micron pore size bottle top filter (Corning cat. #430624). Fetal bovine serum was then added to 10% final concentration.

To prepare treated and control media for growth of the MCF-10A cells, F-12 nutrient mix powder (Life technologies cat. #21700-026) was re-suspended in either dc-DEP EMF-treated saline, or with an aliquot of the same solution prior to treatment with the apparatus. The F-12 media was then mixed 1:1 with DMEM prepared as described above with either dc-DEP EMF-treated or control saline and then the DMEM/F-12 was supplemented with EGF, cholera toxin and insulin as described above and then filter sterilized as described above. Horse serum was then added to 5% final concentration.

### Cell Growth Studies

Treated groups of MDA-MB231 cells were cultured in the DMEM-10 with media that was reconstituted with a hypotonic saline solution that had been treated with the dc-DEP force EMF for 30 minutes and the control groups were cultured in media that was reconstituted with the same hypotonic saline solution prior to treatment with the BFA system. The treated group of MCF-10A cells was cultured in the DMEM/F12-5 media that had been reconstituted with a hypotonic saline solution that had been treated with the dc-DEP force EMF for 30 minutes and the control group was cultured in media that was reconstituted with a hypotonic saline solution that had not been treated with the dc-DEP force EMF. On day one, aliquots of 10,000 cells were plated in three 6-well plates for each of the two groups for each of the two cell lines. They were plated in their standard (non-EMF treated) DMEM-10 or DMEM/F12-5 media on day 1. On day 2, the treated (n=21) and control (n=21) media for each of the two cell lines were made and the original standard media was replaced in each of the wells with the newly prepared treated and control media. On days 3 through 7, media was prepared and changed daily and wells from the control group and treated group of each cell lines were trypsinized, removed from 3 wells of each group and counted and cell size of each sample was measured using a Scepter cell counter (EMD Millipore). Growth data in the growth studies was analyzed with Mann Whitney U since the tests for normality were not met by a bell-curve distribution upon histogram analysis.

### Mitotic Index by Tubulin Staining

Replicate 60 mm dishes of MDA-MB231 cells (6 plates of each control and treated) were plated in standard DMEM-10 media. On day 2 the media in the control and treated groups were replaced and the cells were allowed to grow for 1 day. On day 3 the cells were washed with PBS and fixed with paraformaldehyde. The cells were then stained with DAPI (4′, 6-diamidino-2-phenylindole) to stain the DNA blue. Then an anti-tubulin stain was added prior to staining the cytoskeleton/microtubules with rhodamine—conjugated secondary antibody (red). Cells were then examined with fluorescent microscopy and percent cells seen in the different stages of mitosis were calculated to achieve a mitotic index.

### Microarray Analysis

Replicate 60 mm dishes of either MDA-M13-231 or MCF-10A (5 plates each for growth in treated and control media) were plated in DMEM-10 and the next day the media were replaced with either treated or control media which was replaced daily with freshly prepared treated or control media for the next two days. On day 4 post-plating (day 3 post-treatment) the cells were removed with trypsin, counted and 3×10⁶ cells from each plate were collected by centrifugation and total RNA was isolated using the RNeasy Mini Kit according to the manufacturer's instructions (Qiagen). RNA concentration was determined and RNA integrity was evaluated using an Agilent 2100 Bioanalyzer (Agilent Technologies) and all RNA integrity number (RIN) values were ≥10. The RNAs from the five biologic replicates from each group were combined and cDNA was generated using Ambion WT amplification kit (ThermoFisher Scientific) according to the manufacturer's instructions. The samples were subsequently fragmented and labeled using the Affymetrix WT Terminal Labeling kit and then hybridized, together with the probe array controls, onto the Human Genome U133 Plus 2.0 GeneChip Array (Affymetrix). The array was washed and stained using an Affymetrix Fluidics Station 450, scanned on an Affymetrix GCS3000 7G scanner, and the data were normalized by Robust Multichip Averaging (RMA) using the Affymetrix expression console in order to transform all the arrays to have a common distribution of intensities by removing technical variation from noisy data before analysis. To quantile normalize two or more distributions to each other, both treated and control groups were set to the average (arithmetical mean) of both distributions. Therefore, the highest value in all cases becomes the mean of the highest values, the second highest value becomes the mean of the second highest values.

### Quantitative RT-PCR

Hyaluronan-mediated motility receptor (HMMR) showed significant differences in expression between the treated and control MDA-MB-231 groups in the microarray analyses and these were chosen for validation by reverse transcription-quantitative polymerase chain reaction (RT-qPCR). Ribosomal protein S19 (RIBOPROTS9) was used for normalization. The primers were designed using the Universal Probe Library Probe Finder assay design software (Roche) and sequences are available upon request. RNAs from the 5 biological replicates were reverse transcribed individually using the Transcriptor First Strand cDNA Synthesis Kit (Roche) to generate cDNAs according to the manufacturer's protocol. Ten-fold serial dilutions (10⁻¹-10⁻⁴) of each of the cDNAs were then mixed with the appropriate universal library probe (UPL probe, Roche), sense and anti-sense primers, and reaction buffer into 96-well plates. The polymerase was activated by incubation at 95° C. for 5 minutes followed by a 45-cycle amplification consisting of denaturation at 95° C. for 15 seconds, annealing at 60° C. for 1 minute and elongation at 72° C. for 5 minutes.

### Results

### Cell Growth and Mitotic Spindle

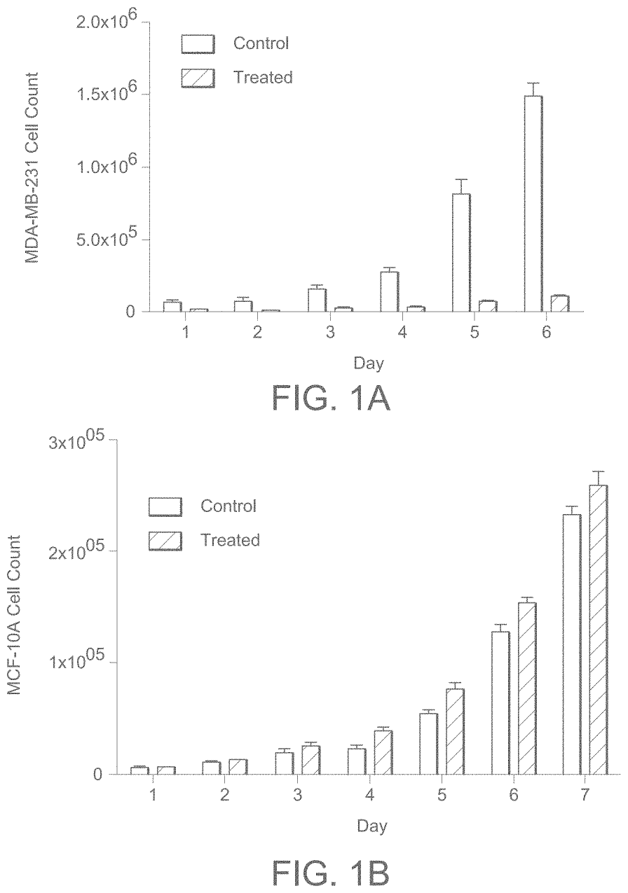

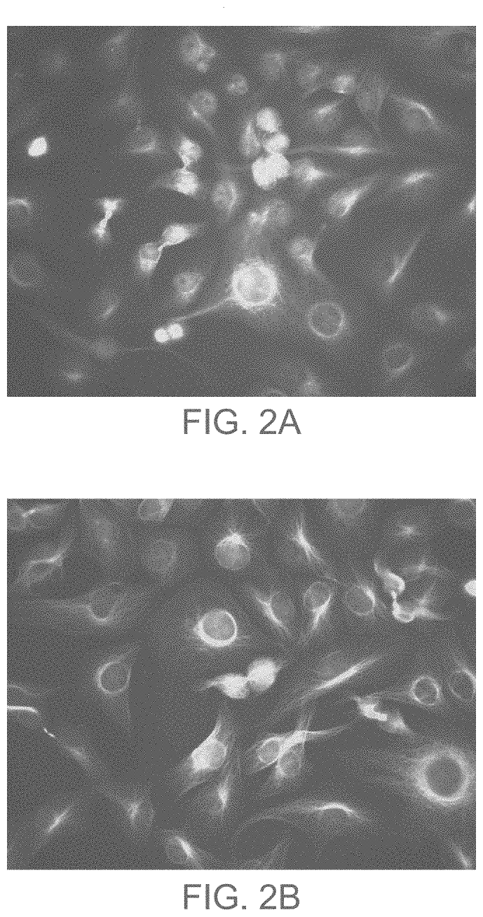

Initial experiments showed significant growth inhibition of the human breast carcinoma when grown in the dc-DEP EMF force treated media when compared to the control group which showed no growth inhibition, as shown in FIG. 1A. Alternatively, the human breast epithelial cells showed no growth inhibition between the control and treated groups, as shown in FIG. 1B. After 1 day of growth in the treated media, the tubulin staining of the human breast carcinoma showed a significant difference in the mitotic spindle formation between the control and treated groups. The control group showed approximately 20% of the cell undergoing mitotic spindle formation while 0% mitotic spindle formation was noted in the treated groups, as shown in FIG. 2.

### Microarray and RT-qPCR

Microarray analysis of the control versus treated groups of the human breast carcinoma identified 1,165 genes that were up-regulated over 2-fold and 872 genes that were down-regulated over 2-fold in the treated groups grown in the media that was reconstituted with the dc-DEP force EMF-treated hypotonic saline solution. For comparison, 431 transcripts showed over 2-fold changes in the dc-DEP force EMF-treated versus non-treated human epithelial cells. One of the genes that was noted to be significantly changed between the treated and control groups of the human breast carcinoma in the microarray analyses was HMMR. HMMR showed significant down regulation by RT-qPCR via unpaired t-tests (Table 1) along with a 16-fold decreased in expression by analysis of the delta-delta CT method in the treated group. There was no significant change in gene expression by the unpaired t-tests between the treated versus control groups of the MCF-10A epithelial cells (Table 1).

TABLE 1
Unpaired T-Tests of RT-qPCR for HMMR Expression between Treated
and Control Groups of MDA-MB231 cells and MCF-10A cells.

| | Unpaired t-Tests | | | | |
|---|---|---|---|---|---|
| Gene | df | ts | P | Mean/Control | Mean/Treated |
| MDA-MB231 | | | | | |
| HMMR | 8 | 6.38892 | .000212 | 6.744648 | 10.32 |
| MCF-10A | | | | | |
| HMMR | 8 | 0.05803 | .955132 | 12.667 | 12.677 |

df- degrees of freedom;
ts- test statistic;
p- p value

### Discussion

The experiments using the dc-DEP EMF application with the MDA-MB231 cells showed a significant growth inhibition, as depicted in FIG. 1, and changes in mitotic spindle formation, as depicted in FIG. 2, when these cells are grown in the dc-DEP EMF treated growth media when compared to controls. There have also been changes in chloride ion channel expression and cell polarity changes with this dc-DEP EMF force application from the BFA apparatus (Purnell et al., 2016). The significant down regulation of HMMR in our RT-PCR analysis when coupled with the decreased cell growth/mitosis and decreased mitotic spindle formation in the treated versus control human breast carcinoma suggests that a decreased cell motility/migration/metastasis could occur when the cancerous cells are exposed to the dc-DEP EMF force. Interestingly, there was no decreased growth/mitosis noted in the human breast epithelial cells and HMMR showed no significant change in the human breast epithelial cells in our microarray analyses.

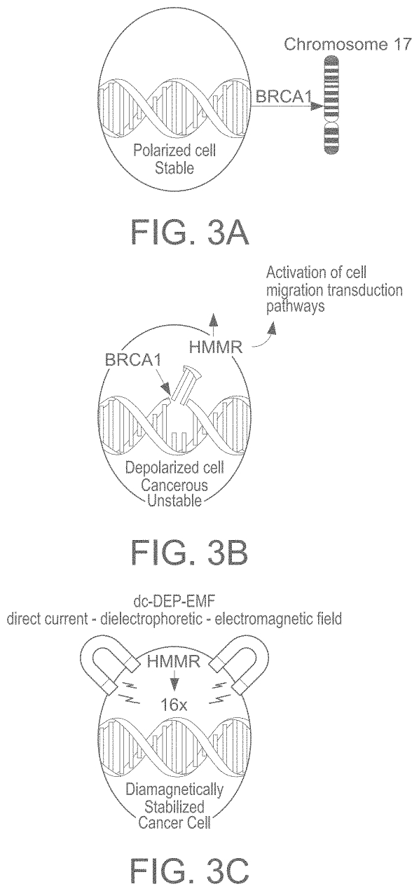

Since the dc-DEP EMF produced by the BFA has hyperpolarized both human breast carcinoma and human breast epithelial cells in vitro (Purnell et al., 2016), it is further contemplated that applied dc-DEP EMF may offer the polarization stability that is lacking with both the inherited BRCA1 mutations and other non-inherited cancers. Initiation of cell migration pathways occurs when a cell/cells are damaged or depolarized and these same mechanisms that spark wound healing may initiate tumor cell metastasis. It is contemplated that the dc-DEP EMF force that is generated by the BFA apparatus differentially enhances wound healing and cell migration in noncancerous cells while inhibiting cell migration/metastasis in cancerous cells. Control of HMMR expression through the application of this dc-DEP EMF force is a contemplated application of this invention to the tumor microenvironment that allows for control of tumor development, progression and metastasis by maintaining cell polarity in the presence of BRCA1 mutations and other depolarized cell states, as shown in FIG. 3C.

### CONCLUSION

The dc-DEP EMF force leads to a significantly decreased expression in HMMR in human breast carcinoma and offers a novel approach to developing an understanding of the relationship between the tumor microenvironment and tumor initiation and progression. Even though the mechanisms that link cell migration/wound healing and cell migration/cancer metastasis pose an example of how challenging the development of side effect free cancer treatments can be, this study provides a potential mechanism to stop cancer metastasis without effecting the healing in normal cell migration and repair. Specifically, this study teaches the ability to control cell polarity and HMMR expression under the influence of C on cell membrane diamagnetic anisotropy that is generated by this dc-DEP EMF force, as shown in FIG. 3C. The dc-DEP EMF shows the possibility of offering changes in epigenetic expression in the presence of both inherited genetic mutations (BRCA1) and non-inherited mutations that are involved in tumor progression.

RHAMM has been linked to both inherited and non-inherited based tumor progression. It is contemplated to use this RHAMM link through dc-DEP EMF force to treat cancer patients with this dc-DEP EMF of the BFA apparatus in the form of a footbath/bath. In an embodiment of this invention, the footbath/baths would be given every other day, in addition to the current standard of care, and could be done in the home of the patient as well as in cancer outpatient clinics. This dc-DEP EMF footbath application of the BFA apparatus through immersion therapy (all or part of the organism in contact with the electrolyte solution with the active direct current) offers a new adjunct treatment as well as an alternative to CPM and other surgical interventions for both human and animal patients with inherited and non-inherited aggressive carcinomas.

In another embodiment of this invention, the electrolyte solution actively treated with the BFA apparatus is used as an indirect application (used for a set time/protocol after the direct current is removed) through an (IV) intravenous infusible or PO (by mouth) ingestible for the possible adjunct treatment of cancer and cancer metastasis.

### Bio-Field Array Apparatus

Also provided herein are improvements to the apparatus disclosed in U.S. Pat. No. 6,555,071 B2, incorporated herein by reference, for an apparatus that generates a dielectrophoretic electromagnetic field driven by direct current in a hypotonic electrolytic solution. The bio-field array apparatus, through a direct current driven dielectrophoretic electromagnetic field in a hypotonic electrolyte solution, modulates the membranes of all living cells (plants, animals and humans) and facilitates movement of ions and other critical cell physiological components to allow the cells in an organism to function more efficiently. The apparatus can be used directly or indirectly. An embodiment of the invention includes a direct application having the organism in direct contact with the hypotonic electrolyte solution for a set amount of time at a set frequency while the active direct current was being applied to the electrolyte solution. In another embodiment, the indirect applications include the hypotonic electrolyte solution used after the field has been applied for a set amount of time at a set frequency to, in or for a living organism.

The preferred embodiment of this apparatus uses ˜2.5 to 3.0 amperes of direct current (de) from a power supply that delivers the current to an array (of conductive rings) that when placed into a 3 mM hypotonic saline solution creates a dielectrophoretic (DEP) electromagnetic field (EMF) with a magnetic field intensity of 10⁻³ gauss or 100 nT.

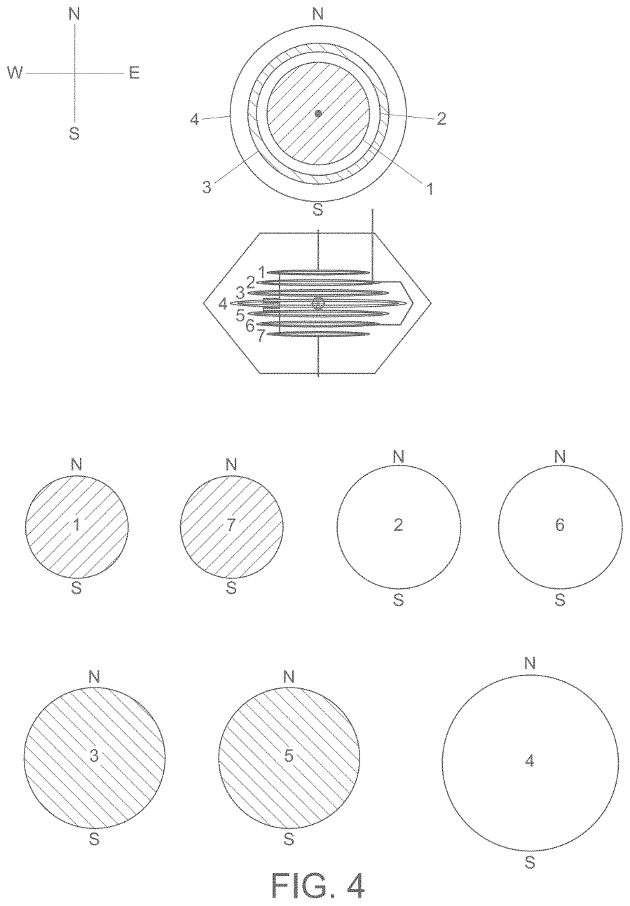

The electrically conductive rings are arranged in a spherical array parallel to one another and can be formed from stainless steel or other similar metals with the central ring being but not limited to a different material such as copper, as shown in FIG. 4. The central ring has a neutral potential and the remaining rings have opposing potentials applied to them by way of appropriate conductive tracks. There are essentially 5 electrically conductive rings and two conductive plates/buttons. The middle ring has the largest diameter and the remaining rings decrease in diameter by pre-determined amounts and in paired sets of the same diameter. In an embodiment of the invention, the diameters adhere to the ratios of the center ring is 79 mm which will set the diameters of the remaining pairs of rings at 66 mm, 53 mm and 40 mm with spacing between the rings of 5 mm.

The non-conductive support housing can be arranged within an orbital covering to protect the apparatus. The housing can be made of a material such as ABS plastics or the like. The power supply uses 110-220 volt AC to 30 volt DC transformer which applies the appropriate potential between the rings.

### Improvements to the Prior Art Apparatus

In one aspect of the prior art, the stainless steel metal sheets used to make the stainless steel metal rings undergo cold working through roll forming to set the magnetic permeability and magnetism of the whole metal sheet by allowing the austenite (gamma iron) to be converted to ferromagnetic martensite or ferrite forms of iron. Our improvements include laser cutting or punching and marking the rings on the polar coordinates (N, S, E, W) that will be identified using a compass after the rings are constructed. Since the compass needle is a small magnet, the South end will be attracted to the North Pole of the magnetized stainless steel metal sheet. Consequently, the rings from the same metal sheet must be used in the same array and must be set to the same polar coordinate position respective to each of the other stainless steel rings. This amended process increases the strength, decreases the noise of and the energy requirements to power the magnetic bio-field since the stainless steel metals will be layered in sync within their native polar coordinates therefore allowing them to operate as their own uniform field within the direct current driven dielectrophoretic electromagnetic field of the entire array. The center non-magnetic metal neutral rings will then be used within these uniformly polarized stainless steel rings. These non-magnetic metal central rings can be made of several different non-magnetic metals that include but are not limited to copper.

In another embodiment, the addition of crystalline piezoelectric materials placed in the center of the array (in the middle of the central copper/neutral ring) influence the bio-field that is emitted by the bio-field array apparatus. The piezoelectric effect occurs when materials exhibiting the direct piezoelectric effect ((generation of an electrical charge (on the crystalline material/membrane potential)) resulting from a mechanical force) also exhibit a piezoelectric effect ((internal generation of a mechanical strain (crystalline material structural shift/membrane chemical shift) resulting from an applied field)). Crystals of piezoelectric material in this improved BFA allow further frequency modulation capabilities of the negative or diamagnetic field of the organism when they are added to the direct current driven dielectrophoretic electromagnetic field of the array. The ability to modulate frequencies with the addition of differing crystalline piezoelectric materials leads to the development of but not limited to enhanced anti-inflammatory and anti-microbial applications. It is contemplated that this ability provides an adjunct and alternative treatment to the current antimicrobial and other chronic disease therapies. While crystals are the preferred stone, quartz and other precious stones added to the center orb for dB/Hz enhancements are contemplated herein.

In yet another embodiment, varying the frequencies powering the array through a power supply is provided where either the poles and/or the hertz frequencies may be adjusted or re-programmed at or from the power supply. These differing hertz frequencies will be tested with protocols based on the improvements discussed elsewhere in this document. These varying hertz frequencies will offer differing frequencies that will be found to correspond to the development of certain signals that are specific to treatment of certain and specific disease. For example, hertz frequencies are developed and programmed into the array to treat specific health and performance issues such as, but not limited to, hypertension, cancer, wound care, autism, Alzheimer's, Parkinson's, organ dysfunction, and athletic stamina. The improvements contemplated herein also include altering the voltage emitted from the apparatus and altering the hertz levels that are sent to the array/orb in the electrolytic solution.

In yet another embodiment, the connecting cord that attaches to the array from the power supply or other member associated with the apparatus is equipped with feedback and sensing capabilities to measure bioimpedance, phase angle and any other frequency data that is emitted by person, animal or organism that is in contact with the water during the BFA treatment and can therefore be measured and quantified. The monitoring of these data allows for further frequency programming from the previously discussed improvements.

Each organism, animal, plant, or human has a DNA signature frequency electromagnetic field. These fields vary from differing states of health and change through the aging process. The ability to adjust the frequency through the re-programming of frequencies from the power supply as well as changing the field with crystalline materials of piezoelectricity that is based on measuring the frequencies, bioimpedance, phase angle and another other electromagnetic signal being emitted from the organism, animal, human or plant in contact with the BFA can have powerful effects on states of health and treatment of chronic disease.

It is contemplated that the BFA can be used to actively treat humans and animals (organism has part, most or all of their body immersed in the electrolyte solution) for a set amount of time at a set frequency with the active direct current flowing to the BFA. The living organism can also be in contact with the treated water for a set time after the current has been removed to receive the field effects of the water.

It is further contemplated that the apparatus is used to treat a hypotonic electrolyte solution for a set amount of time and at a set frequency which can then be used to water plants, reconstitute differing growth mediums for living things (in vitro and in vivo), generate drug delivery systems, generate intravenous (IV) applications for humans and animals, generate PO (by mouth) ingestible applications for humans and animals.

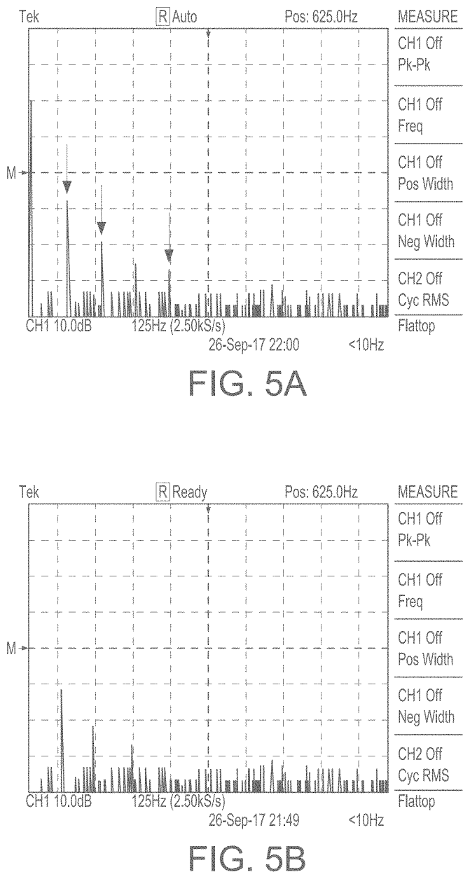

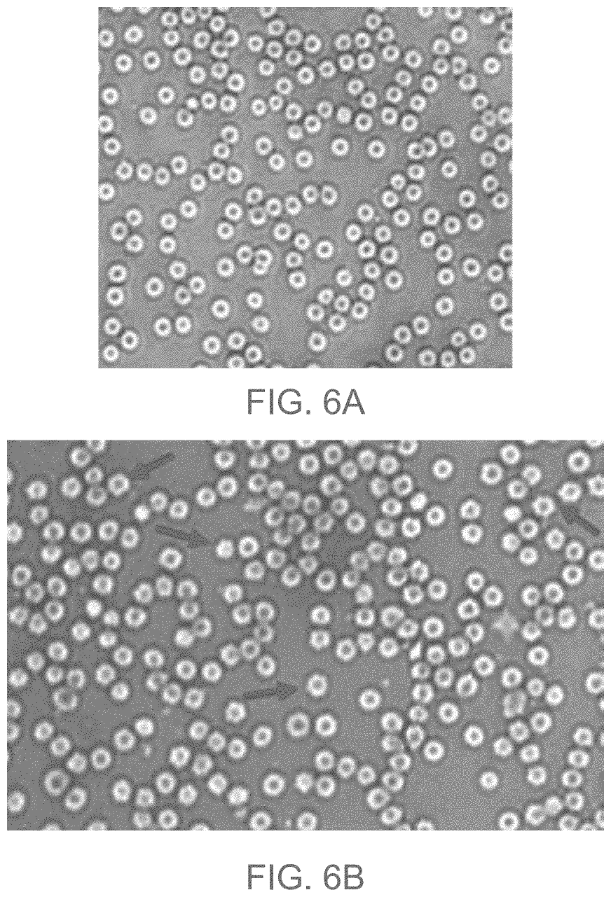

The oscilloscope depicted in FIGS. 5A-5B shows the decibel/Hertz readings from these above listed improvements to prior art with the BFA. There is the potential to adjust the decibel/Hertz frequency and strength with these changes that will affect differing effects on cell physiology in living organisms. As previously discussed, studies have shown that sound (dB/Hz) traveling through cells creates a kind of mechanical stress that leads to transcriptional and post transcriptional level responses. The red blood cell figures depicted in FIG. 6A-6B show the difference in the red blood cell membranes from the mechanical stress. When the signal is more steady and rises above the ‘noise’ and plays the ‘notes’ more effectively, there is less noise that reaches the membrane and allows for a more synchronistic field in organics.

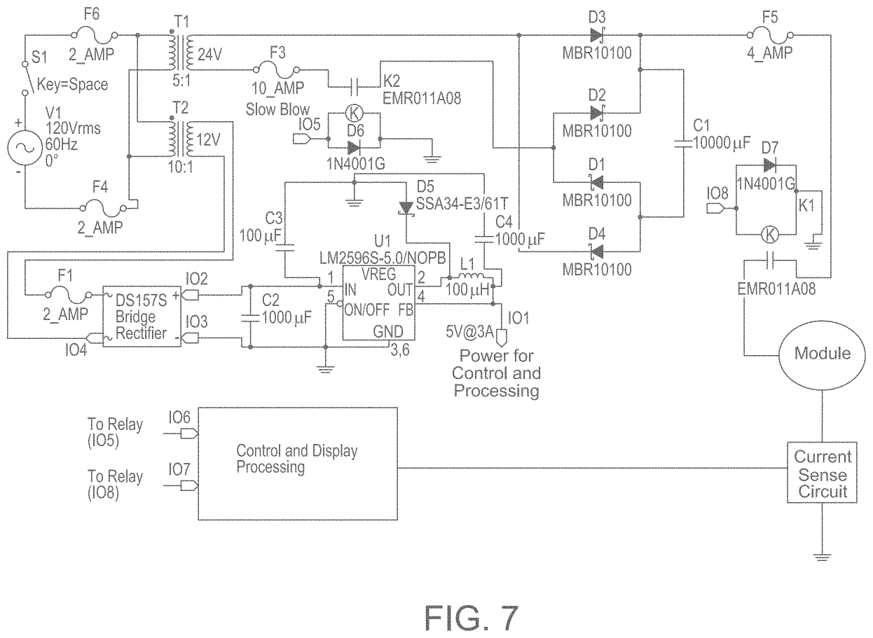

Yet another embodiment of the improved BFA apparatus includes upgrading the standard transformers to toroidal transformers, as shown in FIG. 7. Since the toroid is a closed-loop core, it will emit a higher magnetic field and therefore a higher inductance and Q factor than an inductor of the same value with a straight core (solenoid coils). The toroidal transformer, due to the fact that the windings are relatively short, will have a lower secondary impedance that will increase the efficiency and the electrical performance.

The terms “comprising,” “including,” and “having,” as used in the claims and specification herein, shall be considered as indicating an open group that may include other elements not specified. The terms “a,” “an,” and the singular forms of words shall be taken to include the plural form of the same words, such that the terms mean that one or more of something is provided. The term “one” or “single” may be used to indicate that one and only one of something is intended. Similarly, other specific integer values, such as “two,” may be used when a specific number of things is intended. The terms “preferably,” “preferred,” “prefer,” “optionally,” “may,” and similar terms are used to indicate that an item, condition or step being referred to is an optional (not required) feature of the invention.

The invention has been described with reference to various specific and preferred embodiments and techniques. However, it should be understood that many variations and modifications may be made while remaining within the spirit and scope of the invention. It will be apparent to one of ordinary skill in the art that methods, apparatus, apparatus elements, materials, procedures and techniques other than those specifically described herein can be applied to the practice of the invention as broadly disclosed herein without resort to undue experimentation. All art-known functional equivalents of methods, apparatus, apparatus elements, materials, procedures and techniques described herein are intended to be encompassed by this invention. Whenever a range is disclosed, all subranges and individual values are intended to be encompassed. This invention is not to be limited by the embodiments disclosed, including any shown in the drawings or exemplified in the specification, which are given by way of example and not of limitation.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

All references throughout this application, for example patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application (for example, a reference that is partially inconsistent is incorporated by reference except for the partially inconsistent portion of the reference).

I claim:

1. A method for treating cancer and cancer metastasis comprising exposing cells to a direct current (dc) dielectrophoretic (DEP) electromagnetic field (EMF) force to down regulate Hyaluronan mediated motility receptor (HMMR).

2. The treatment of claim 1 wherein the exposure to dc-DEP EMF force is via a bio-field array apparatus.

3. The treatment of claim 2 wherein the exposure to dc-DEP EMF force is through a direct current driven dielectrophoretic electromagnetic field in a hypotonic electrolyte solution.

4. The treatment of claim 2 wherein the electrolyte solution is actively treated with the bio-field array apparatus through an intravenous (IV) infusible.

5. The treatment of claim 2 wherein the electrolyte solution is actively treated with the bio-field array apparatus as an ingestible.

6. The treatment of claim 2 wherein the bio-field array apparatus is a bath.

7. The treatment of claim 1 wherein the exposure is through immersion therapy.

8. The treatment of claim 1 wherein the cells are cancerous.

9. The treatment of claim 1 wherein the cells are human.

10. The treatment of claim 1 wherein the cells are animal.

11. The treatment of claim 1 wherein the treatment is an adjunct treatment.

12. The treatment of claim 1 wherein the exposure enhances cell polarity.

13. An improved device for generating dielectrophoretic electromagnetic fields in solution comprising a bio-field array apparatus and feedback and sensing capabilities to measure impedance.
a. The device of claim 13 wherein the poles and/or the hertz frequencies are adjusted or re-programmed at or from the power supply.

14. An improved device for generating dielectrophoretic electromagnetic fields in solution comprising a bio-field array apparatus and crystalline piezoelectric materials in the center of the array.

15. An improved device for generating dielectrophoretic electromagnetic fields in solution comprising a bio-field array apparatus and a toroidal transformer for power supply.

16. An improved device for generating dielectrophoretic electromagnetic fields in solution comprising a bio-field array apparatus and the ability to adjust or reprogram the poles and/or the hertz frequencies.

17. The device of claim 16, wherein the hertz frequencies correspond to signals for treatment of diseases.
